(19) United States
(12) Patent Application Publication
(10) Pub. No.: US 2004/0186427 A1
(43) Pub. Date: Sep. 23, 2004

(54) Safety syringe

(76) Inventor: Geo Seong Pok, Songpa-ku (KR)

Correspondence Address: Shlesinger Arkwright & Garvey, 3000 South Eads Street, Arlington, VA, 22202, US

(21) Appl. No.: 10/480,655

(22) PCT Filed: Dec. 20, 2002
(86) PCT No.: PCT/KR02/02400

(30) Foreign Application Priority Data
Jan. 28, 2002 (KR) 10-2002-004870
Oct. 4, 2002 (KR) 10-2002--060575

(51) Int. Cl. A61M 5/00

(52) U.S. Cl. 604/110; 604/218

(57) ABSTRACT

Disclosed herein is a safety syringe. According to the safety syringe of the present invention, an adapter connected to an injection needle is threadedly coupled with the neck of a cylinder while interposing an O-ring therebetween. Thus, the movement of the injection needle is effectively prevented while in use, and thus the injection needle can be pricked accurately at a desired position of the patient's body. Since the adapter is formed with grooves, whereas a plunger is formed with two protrusions positioned diametrically opposite to each other so as to be inserted into the grooves, the adapter is rotated and then moved downwardly in accordance with the successive rotation and downward movements of the plunger after the adapter is released from the neck of the cylinder. The plunger is formed at its upper surface with a vertical rod extending upwardly from an eccentric position, whereas the adapter is defined with a conical cavity. The upper end of the rod is inserted into the cavity while being bent inwardly. When the injection needle is withdrawn into the cylinder, the injection needle is firmly tilted in a lateral direction due to the strong restoring force of the rod so that it can be collapsed simply against the inner surface of the cylinder. In addition, a circular plate is formed at a certain lower position of the plunger. The circular plate enables the user's fingers gripping the safety syringe not to come into contact with the plunger to be entered into the cylinder, thereby achieving the sanitary use of the safety syringe.

### TECHNICAL FIELD

The present invention relates to a syringe, and more particularly to a safety syringe which is configured to allow an injection needle to be withdrawn into a cylinder and then collapsed after injection of liquid medicine, thereby preventing reuse of the syringe, protecting medical personnel and others from being stuck by the injection needle after completion of injection, and avoiding safety-related accidents.

### BACKGROUND ART

There is known in the art a safety syringe which is configured to allow an injection needle to be withdrawn into a cylinder and then collapsed, after injection of injection liquid.

A conventional safety syringe comprises an elongate cylinder, an adapter, a hub, an injection needle and a plunger. The elongate cylinder defines a space for accommodating the injection liquid and is provided with a neck extending upwardly from the upper end thereof. The adapter is vertically fitted and coupled into the neck of the cylinder with interposing a sealing ring therebetween. The hub is fixed to the upper end of the adapter, and the injection needle is fixedly fitted through the hub. The plunger is inserted inside the cylinder so as to be movable upwardly and downwardly. The plunger is adapted to suck the injection liquid into the cylinder and to discharge the injection liquid accommodated within the cylinder to the outside. The plunger is provided at its upper surface with a projection extending upwardly from the plunger. The projection has the same central axis as a central axis of the cylinder and is formed at its upper end with an arrow-shaped head. The adapter is formed with a socket eccentrically positioned from the central axis of the cylinder. The arrow-shaped head formed at the projection of the plunger is adapted to be engaged into the socket of the adapter.

Now, the using method of the conventional safety syringe constructed as stated above will be described. Where the plunger of the safety syringe is fully inserted into the cylinder, the arrow-shaped head formed at the projection of the plunger is inserted through the socket of the adapter and coupled thereto at the same time with the completion of injection. In this state, as the plunger is withdrawn and pulled out of the cylinder, the adapter, hub and injection needle affixed to the adapter are also withdrawn into the cylinder. In a state that the injection needle is completely withdrawn into the cylinder, the injection needle is tilted laterally inside the cylinder due to the presence of the socket of the adapter eccentrically positioned with respect to the cylinder. After that, when the plunger is pushed again into the cylinder, the injection needle is bent and collapsed within the cylinder. Thus, it is possible to prevent reuse of the safety syringe.

However, the conventional safety syringe constructed as stated above suffers from a defect in that the injection needle moves unstably when an excessive force more than a predetermined magnitude is applied thereto, since the injection needle is fitted through the neck of the cylinder while being movable vertically and linearly. For this reason, it is difficult to prick the injection needle at a precise position into the patient's body. There is another disadvantage of increased manufacturing cost of the safety syringe due to the complicated shape of the arrow-shaped head formed at the projection of the plunger to be engaged into the socket of the adapter. Furthermore, in a state that the adapter is firmly coupled to the neck of the cylinder under the application of excessively large force, the arrow-shaped head formed at the projection of the plunger is disengaged from the socket of the adapter as the plunger is pulled out of the cylinder. This causes the adapter, the hub and injection needle attached to the adapter to fail to be withdrawn into the cylinder.

At the time that the injection of the injection liquid is completed, a shock is generated and transferred to the patient's body when the arrow-shaped head formed at the projection of the plunger is forcibly engaged into the socket of the adapter.

The conventional safety syringe has a somewhat complex construction in that the injection needle thereof is fixedly fitted through the hub and again the hub is fixed to the adapter. This results an increase in manufacturing cost of the safety syringe.

Since the user's fingers gripping the safety syringe come into contact with the plunger entering into the cylinder, there is a contamination risk due to the input of impurities into the patient's body.

### DISCLOSURE OF THE INVENTION

Therefore, the present invention has been made in view of the above problems, and it is an object of the present invention to provide a safety syringe which is configured so that an injection needle is fixedly fitted in an adapter and the adapter is firmly coupled to a cylinder, thereby preventing the injection needle from moving unstably when in use, and allowing the injection needle to be simply withdrawn into the cylinder and then collapsed therein after completion of injection.

It is another object of the present invention to provide a safety syringe which is configured so that an adapter, in which an injection needle is fixedly fitted, is firmly coupled to a cylinder after a plunger is fully pushed into the cylinder, thereby allowing the adapter to be reliably withdrawn into the cylinder after completion of injection.

It is another object of the present invention to provide a safety syringe which is configured to minimize the amount of shock transferred to the patient' body when injection of liquid medicine is completed.

It is another object of the present invention to provide a safety syringe which is configured to allow an injection needle to be tilted laterally inside a cylinder by a large eccentric force applied thereto when the injection needle is withdrawn into the cylinder, thereby reliably preventing the injection needle from falling out of the cylinder.

It is another object of the present invention to provide a safety syringe which is configured to reduce a manufacturing cost by simplifying the coupling structure of an injection needle.

It is further object of the present invention to provide a safety syringe which is configured to prevent the user's fingers from coming into contact with a plunger to be pushed into a cylinder, thereby preventing the introduction of impurities into the patient's body.

In accordance with the present invention, the above and other objects can be accomplished by the provision of a safety syringe comprising: a cylinder defining a space for accommodating injection liquid and having a neck extending upwardly from an upper end thereof; a plunger inserted inside the cylinder while being movable in the cylinder; an adapter coupled to the neck of the cylinder; and an injection needle connected to the adapter, wherein: the plunger is provided at its upper surface with a vertical rod extending upwardly from an eccentric position with respect with a central axis of the cylinder; the adapter is defined at its interior space with a cavity, into which the rod is inserted while being bent inwardly at the upper end thereof in accordance with the upward movement of the plunger; the adapter is threadedly coupled to the neck of the cylinder; and coupling means are formed at the adapter and plunger, respectively, and adapted to allow the adapter to be rotated and then moved downwardly in accordance with the successive rotating and downward movements of the plunger, thereby causing the injection needle to be withdrawn into the cylinder.

Preferably, the coupling means may comprise a plurality of grooves defined at an inner surface of and adjacent to a lower end of the adapter and protrusions extending upwardly and outwardly from the upper surface of the plunger and adapted to be inserted into the grooves.

Preferably, a first one of the protrusions may be formed at a lower end of the rod, and a second one of the protrusions may be formed at the upper surface of the plunger axially opposite to the first one.

In the safety syringe of the present invention constructed as stated above, when the plunger is pressed to be fully pushed into the cylinder, the outwardly-extended protrusions formed at the upper surface of the plunger are inserted into the grooves defined at the adapter, and the upper end of the rod is bent inwardly toward the central axis of the cylinder while coming into contact with an inner wall of the cavity defined in the adapter, thereby being inserted into the cavity.

In this state, when the plunger is rotated, the adapter is rotated along with the plunger because the protrusions of the plunger are inserted into the grooves of the adapter. During rotation, the adapter is released from the neck of the cylinder and then the adapter and injection needle are withdrawn into the cylinder by pulling the plunger out of the cylinder. Upon completion of withdrawal, the upper end of the rod, bent inwardly toward the central axis of the cylinder, is vertically straightened and pushes the inner surface of the adapter. According to the strong restoring force of the rod pushing the inner surface of the adapter, the adapter and injection needle attached to the adapter are tilted laterally inside the cylinder. In this state, as the plunger is further pushed into the cylinder, the injection needle is bent excessively and collapsed against an inner surface of the cylinder around an upper portion of the cylinder.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a sectional view illustrating a safety syringe in accordance with a first embodiment of the present invention;

FIG. 2 is an exploded perspective view of the safety syringe shown in FIG. 1;

FIG. 3 is an enlarged sectional view illustrating an adapter and the upper end of a plunger included in the safety syringe shown in FIG. 1;

FIG. 4 is a cross sectional view taken along the line A-A of FIG. 3;

FIG. 5 is a plan view of the plunger shown in FIG. 3;

FIG. 6 is a sectional view of the safety syringe according to the first embodiment of the present invention, illustrating a state in which injection of liquid medicine is completed;

FIG. 7 is an enlarged sectional view of the safety syringe according to the first embodiment of the present invention, illustrating a state in which the adapter and the upper end of the plunger shown in FIG. 6 are coupled with each other;

FIG. 8 is a sectional view of the safety syringe according to the first embodiment of the present invention, illustrating a state in which an injection needle assembly is withdrawn into the cylinder;

FIG. 9 is a sectional view of the safety syringe according to the first embodiment of the present invention, illustrating a state in which an injection needle assembly withdrawn into the cylinder is collapsed inside the cylinder;

FIG. 10 is a sectional view of a safety syringe in accordance with a second embodiment of the present invention;

FIG. 11 is an exploded perspective view of the safety syringe shown in FIG. 10;

FIG. 12 is an enlarged sectional view illustrating an adapter and the upper end of a plunger included in the safety syringe shown in FIG. 10;

FIG. 13 is a cross sectional view taken along the line B-B of FIG. 12; and

FIG. 14 is a cross sectional view taken along the line C-C of FIG. 12.

### BEST MODE FOR CARRYING OUT THE INVENTION

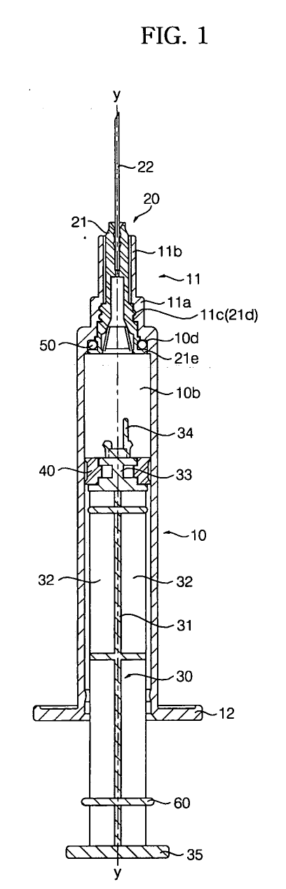

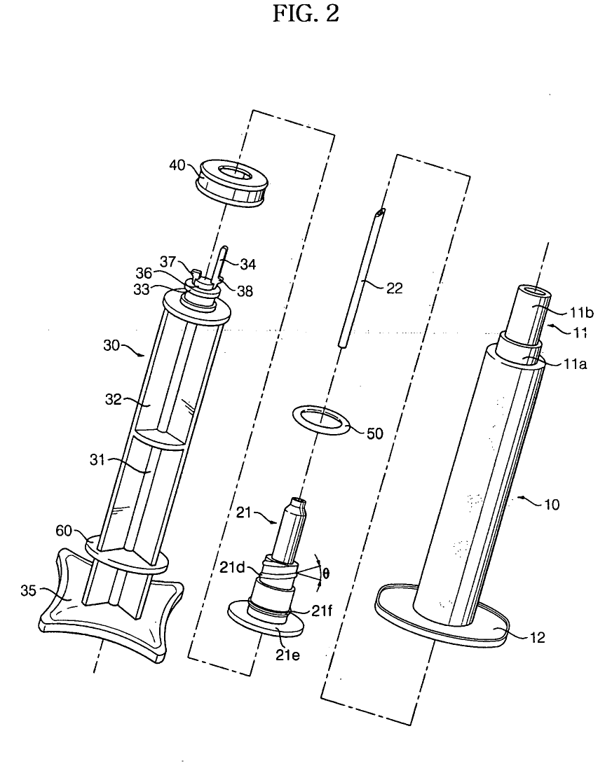

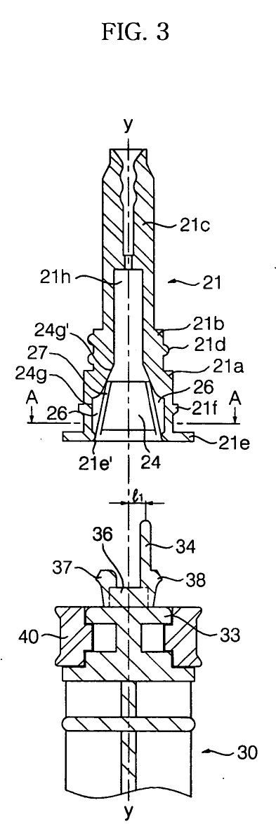

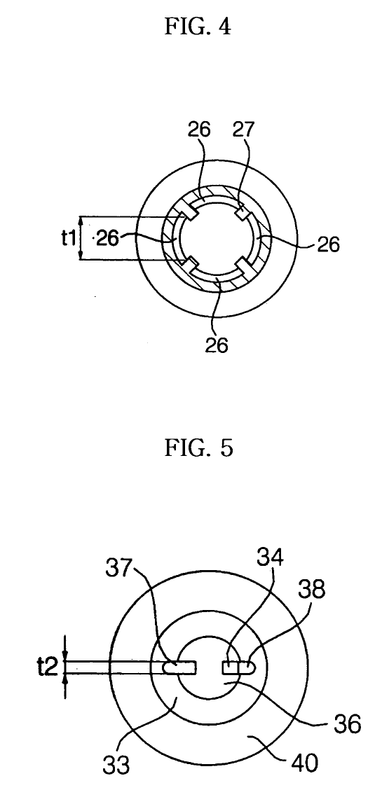

FIG. 1 is a sectional view illustrating a safety syringe in accordance with a first embodiment of the present invention. FIG. 2 is an exploded perspective view of the safety syringe according to the first embodiment of the present invention. FIG. 3 is an enlarged sectional view illustrating an adapter and the upper portion of a plunger included in the safety syringe shown in FIG. 1.

As shown in FIGS. 1 to 3, a safety syringe in accordance with a first embodiment of the present invention comprises an elongate cylinder 10 defining a space 10b for accommodating injection liquid, an injection needle assembly 20 connected to the upper end of the cylinder 10, and a plunger 30 partially inserted into the cylinder 10 while being movable vertically. The plunger 30 is used to suck the injection liquid into the cylinder 10 and to discharge the injection liquid accommodated in the cylinder 10 to the outside.

The cylinder 10 is formed at its upper end with a cylindrical neck 11 extending upwardly from the upper end thereof. The cylindrical neck 11 has a diameter smaller than that of the cylinder 10. Also, the cylindrical neck 11 has a central axis corresponding to a central axis Y of the cylinder 10. The cylindrical neck 11 comprises a lower neck section 11a, and an upper neck section 11b having a diameter smaller than that of the lower neck section 11a. The lower neck section 11a is formed at its inner surface with female threads 11c. The cylinder 10 is formed at its lower end with a handle 12.

The injection needle assembly 20 comprises an adapter 21 to be coupled in the neck 11 of the cylinder 10, and an injection needle 22 attached to the adapter 21 at its lower portion.

As shown in FIGS. 2 and 3, the adapter 21 generally has a cylindrical contour, and comprises a lower cylindrical section 21a to be coupled in the upper end of the cylinder 10, a middle cylindrical section 21b to be coupled in the lower neck section 11a, and an upper cylindrical section 21c to be fitted into the upper neck section 11b. The middle cylindrical section 21b is formed at its outer surface with male threads 21d to be threadedly coupled with the female threads 11c of the lower neck section 11a. The adapter 21 is formed at its lower surface with a flange 21e.

The male threads 21d of the middle cylindrical section 21b and the female threads 11c of the lower neck section 11a are formed as one to eight helical threads, respectively. The helical threads have an inclination angle θ of 25° to 50° measured from a horizontal plane of the flange 21e.

Preferably, the helical threads are formed as two threads and have an inclination angle θ of 30°.

The cylinder 10 is formed at its inner surface with an annular recessed seat 10d adjacent to the upper end thereof. When the adapter 21 is threadedly coupled in the neck 11 of the cylinder 10, the flange 21e of the adapter 21 is fitted into the annular recessed seat 10d of the cylinder 10, and also an O-ring 50 is inserted in the annular recessed seat 10d so that it is located over the flange 21e. In this way, the adapter 21 is firmly coupled to the upper portion of the cylinder 10.

The adapter 21 is formed at the outer surface of the lower cylindrical section 21a with an annular step 21f. The O-ring 50 is fitted between the flange 21e and the annular step 21f.

The plunger 30 comprises a rod-shaped body 31 having a central axis corresponding to the central axis Y of the cylinder 10, and a plurality of ribs 32 radially outwardly extending from the outer surface of the rod-shaped body 31. The rod-shaped body 31 is formed at its upper end with a head 33 and at its lower end with a polygonal knob0 35.

Preferably, in order to ensure that the polygonal knob 35 is easily rotated by the user's fingers, the polygonal knob 35 has a quadrangular contour with its respective side edges dented inwardly.

A circular plate 60 is formed at the rod-shaped body 31 of the plunger 30 while being spaced apart from the knob 35 by a certain distance.

The circular plate 60 has a diameter larger than that of the cylinder 10, so that the circular plate 60 is not allowed to enter into the cylinder 10. Where the user grips the knob 35 of the plunger 30 with his/her fingers to inject the injection liquid, the fingers are supported by the circular plate 60. Thus, it is possible to prevent the user's fingers from coming into contact with a region of the plunger 30 to be pushed into the cylinder 10, and consequently to prevent the safety syringe from being contaminated by impurities such as bacteria.

As shown in FIGS. 2, 3 and 5, the head 33 of the plunger 30 is formed at its upper surface with an elongate vertical rod 34 in the shape of quadrangular prism. The elongate rod 34 extends upwardly from an eccentric position spaced apart from the central axis Y of the cylinder 10 by a distance l₁.

The adapter 21 is defined with a cavity 24 having a certain inner volume extending upwardly from the lower end of the adapter 21. In the lower cylindrical section 21a of the adapter 21, the adapter 21 has a cylindrical inner wall 24g around the lower half portion of the cavity 24, and a conical inner wall 24g′ above the cylindrical inner wall 24g, namely, around the remained upper portion of the lower cylindrical section 21a. The conical inner wall 24g′ has a diameter decreasing gradually toward the upper side thereof. The cavity 24 has a central axis corresponding to the central axis Y of the cylinder 10.

According to its upward movement, the rod 34 of the plunger 30 comes into contact with the conical inner wall 24g′ of the adapter 21 at the upper end thereof. By virtue of the shape of the conical inner wall 24g′, the upper end of the rod 34 is bent inwardly toward the central axis Y of the cylinder 10.

The cavity 24 of the adapter 21 is communicated with an elongate bore 21h positioned above the upper end of the cavity 24, thereby causing the injection liquid to flow from the cavity 24 to the elongate bore 21h.

The plunger 30 and adapter 21 are formed with coupling means, respectively. The coupling means allow the adapter 21 to be rotated and then moved downwardly in accordance with the successive rotating and downward movements of the plunger 30. The coupling means comprise grooves 26 defined on the inner surface of and adjacent to the lower end of the adapter 21 and a pair of protrusions 37 and 38 formed above the head 33 of the plunger 30 and adapted to be inserted into the grooves 26.

A first protrusion 38 extends outwardly from the lower end of the rod 34, and a second protrusion 37 extends outwardly and upwardly from the upper surface of the head 33. The first and second protrusions 38 and 37 are directed on opposite sides of the plunger.

In detail, the second protrusion 37 is formed at one side of a circular extrusion 36 protruded upwardly and outwardly from the head 33 of the plunger 30. The second protrusion 37 is positioned 180° opposite from the first protrusion 38 about the central axis Y of the cylinder 10.

The rod 34 and protrusions 38 and 37 are made from flexible materials so that they are flexibly bent by an external force applied thereto and restore their original straight shape as soon as the external force is removed.

The cavity 24 is formed at its inner surface with four grooves 26 equally spaced from each other. A width t₁ of the respective grooves 26 has a value larger than a width t₂ of the respective protrusions 37 and 38 formed at the plunger 30, so the respective protrusions 37 and 38 are fitted into one of the grooves 26, respectively, in accordance with the upward movement of the plunger 30.

The grooves 26 are defined by four wall portions 27 radially and inwardly protruded from the inner surface of the cavity 24 while being uniformly spaced from each other.

Each groove 26 is formed at its lower end with an annular step portion 21e′ extending inwardly from the flange 21e. The annular step portion 21e′ has a curved inner surface. As the plunger 30 is pushed inside the cylinder 10, although the protrusions 37 and 38 collide against the lower surfaces of the wall portions 27, the respective protrusions 37 and 38 are bent slightly and then inserted smoothly into one of the grooves 26, respectively, while sliding along the curved inner surface of the annular step portion 21e′.

The head 33 of the plunger 30 is covered at its outer surface with an annular packing member 40.

Now, the assembly process and operations of the safety syringe constructed as stated above according to the first embodiment of the present invention will be described. First, the O-ring 50 is fitted around the adapter 21. The adapter 21 fitted with the O-ring 50 is inserted into the cylinder 10 at the lower end of the cylinder 10 toward the neck 11 of the cylinder 10 by a predetermined force applied thereto until the adapter 21 is threadedly coupled with the neck 11. The adapter 21 is further moved upwardly while being threadedly coupled with the neck 11 so that the flange 21e of the adapter 21 is fitted into the annular seat 10d formed at the inner surface of the cylinder 10 adjacent to the upper end thereof. In this way, the adapter 21 is firmly coupled with the neck 11 of the cylinder 10. In this state, the injection needle 21 is inserted into the adapter 21 through a mouth of the adapter 21 and attached to the adapter 21 using an adhesive. Finally, the packing member 40 is covered around the head 33 of the plunger 30 and the plunger 30 is fitted into the cylinder 30. In this way, the assembly process of the safety syringe is completed.

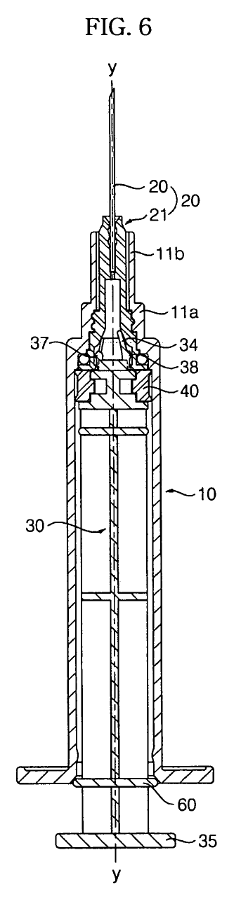

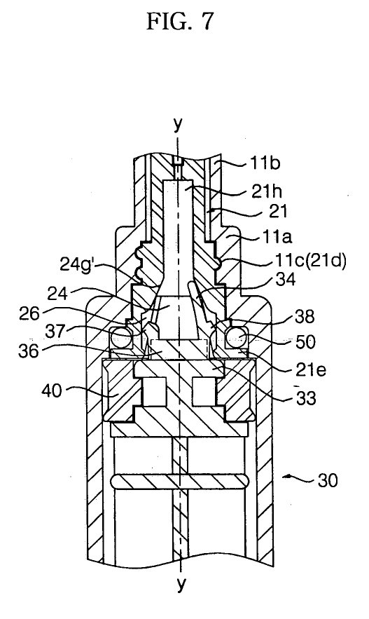

In operation, the plunger 30 is pulled out of the cylinder 10 in a state that the injection needle 22 is inserted in a container receiving the injection liquid to suck the injection liquid and to allow the injection liquid to be accommodated in the space 10b of the cylinder 10. Then, when it is desired to inject the injection liquid to the patient's body, the injection needle 22 is positioned to a desired position of the patient's body and then the plunger 30 is pushed into the cylinder 10 so that the injection liquid charged in the space 10b of the cylinder 10 is injected into the patient's body through the injection needle 22. Then, as the plunger 30 is further pushed into the cylinder 10, the packing member 40 covered around the head 33 of the plunger 30 comes into contact with the flange 21e of the adapter 21, as shown in FIGS. 6 and 7. In this way, the injection operation of the safety syringe is completed.

Upon completion of injection, the respective protrusions 37 and 38 formed at the head 33 of the plunger 30 are inserted into one of the grooves 26 defined at the inner surface of the adapter 21, respectively, and the rod 34 of the plunger 30 is fitted in the cavity 24 of the adapter 21. In this state, the upper end of the rod 34 is bent inwardly toward the central axis X of the cylinder 10 while coming into contact with the conical inner wall 24g′ of the adapter 21. As the rod 34 is bent inwardly, as shown in FIG. 7, the circular extrusion 36 and the second protrusion 37 are bent leftwardly and forcibly pressed against the inner surface of the adapter 21. Upon completion of injection, the injection needle 22 is removed from the patient's body. As the knob 35 of the plunger 30 is reversely rotated, the injection needle assembly 20 including the adapter 21 is also rotated reversely along with the plunger 30 because the protrusions 38 and 37 of the plunger 30 are fitted into the grooves 26 of the adapter 21m, respectively. As a result, the injection needle assembly 20 including the adapter 21 is released from the neck 11 of the cylinder 10, and thus moved slightly in the cylinder 10.

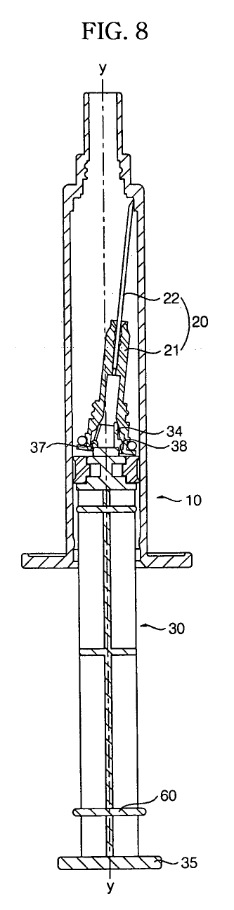

As the plunger 30 and adapter 21 are further rotated integrally, the coupling between the adapter 21 and the neck 11 of the cylinder 10 is completely released. When the plunger 30 is pulled out of the cylinder 10, the protrusions 37 and 38 of the plunger 30 are intercepted by the annular step portion 21e′ of the adapter 21. Thus, the injection needle assembly 20 including the adapter 21, as shown in FIG. 8, is received in the cylinder 10. At this time, the rod 34 of the plunger 30 restores its original vertical position, thereby pressing laterally against the conical inner wall 24g′ of the adapter 21. As a result, the adapter 21 is tilted laterally and thus the upper end of the injection needle 22 comes into contact with the inner surface of the cylinder 10.

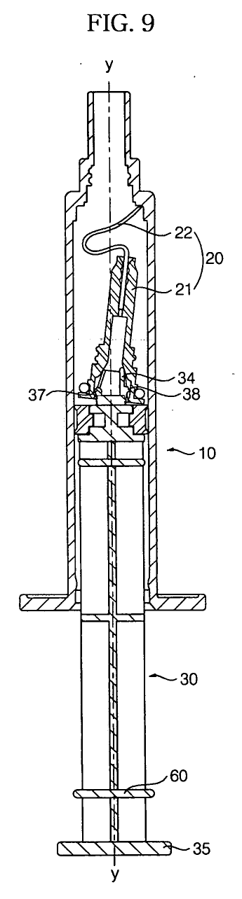

As shown in FIG. 9, as the plunger 30 is again pushed into the cylinder 10, the upper end of the injection needle 22 is bent excessively and collapsed against the inner surface of the cylinder 10 around the upper end of the cylinder 10.

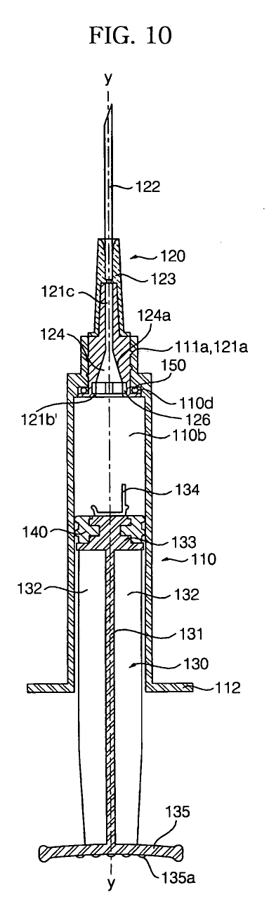

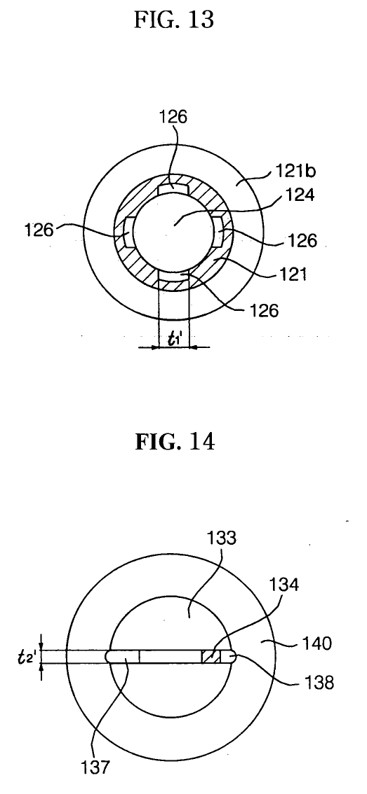

A second embodiment of the present invention is shown in FIGS. 10 to 14.

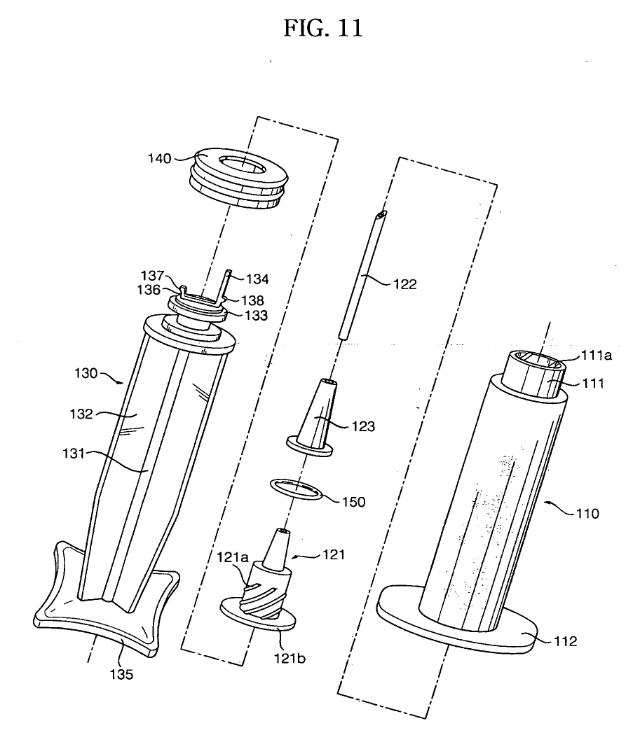

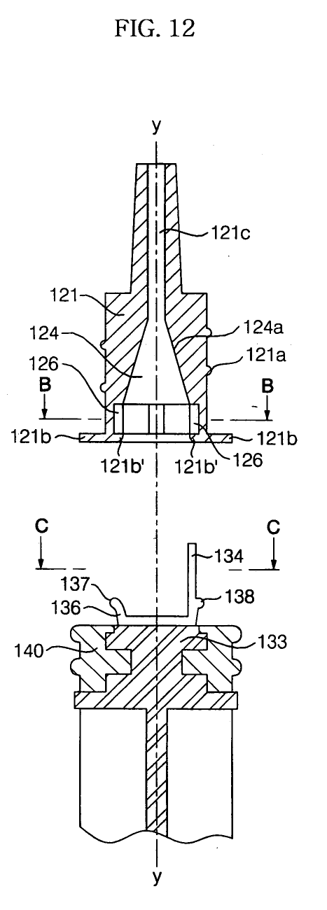

FIG. 10 is a sectional view of a safety syringe in accordance with a second embodiment of the present invention. FIG. 11 is an exploded perspective view of the safety syringe shown in FIG. 10. FIG. 12 is an enlarged sectional view illustrating an adapter and the upper end of a plunger included in the safety syringe shown in FIG. 10.

A shown in FIGS. 10 to 12, the safety syringe in accordance with the second embodiment of the present invention comprises an elongate cylinder 110 defining a space 110b for accommodating injection liquid, an injection needle coupler 120 connected to the upper end of the cylinder 110, and a plunger 130 partially inserted into the cylinder 110 while being movable vertically. The plunger 130 is used to suck the injection liquid into the cylinder 110 and to discharge the injection liquid accommodated in the cylinder 110 to the outside.

The cylinder 110 is formed at its upper end with a cylindrical neck 111 extending upwardly from the upper end thereof. The cylindrical neck 111 has a diameter smaller than that of the cylinder 110. Also, the cylindrical neck 111 has a central axis corresponding to a central axis Y of the cylinder 110. The cylindrical neck 111 is formed at its inner surface with female threads 111a. The cylinder 110 is formed at its lower end with a handle 112.

The injection needle coupler 120 comprises an adapter 121 to be coupled in the neck 111 of the cylinder 110, a hub 123 to be forcibly fitted in the upper portion of the adapter 121, and an injection needle 122 attached to the adapter 121 at its lower portion.

The adapter 121 comprises a lower cylindrical section and an upper conical section. The adapter 121 is formed at its outer surface with male threads 121a to be threadedly coupled with the female threads 111a of the cylindrical neck 111. The adapter 121 is formed at its lower surface with a flange 121b.

The cylinder 110 is formed at its inner surface with an annular recessed seat 110d adjacent to the upper end thereof. When the adapter 121 is threadedly coupled in the neck 111 of the cylinder 110, the flange 121b of the adapter 121 is fitted into the annular recessed seat 110d of the cylinder 110, and also an O-ring 150 is inserted in the annular recessed seat 110d so that it is located over the flange 121b. In this way, the adapter 121 is firmly coupled to the upper portion of the cylinder 110 in an air-tight manner.

The plunger 130 comprises a rod-shaped body 131 having a central axis corresponding to the central axis Y of the cylinder 110, and a plurality of ribs 132 radially outwardly extending from the outer surface of the rod-shaped body 131. The rod-shaped body 131 is formed at its upper end with a head 133 and at its lower end with a polygonal knob 135.

Preferably, in order to ensure that the polygonal knob 135 is easily rotated by the user's fingers, the polygonal knob 135 has a quadrangular contour with its respective side edges dented inwardly. In addition, the polygonal knob 135 is formed at its lower surface with a plurality of fine bosses 135a so as to prevent the slippage of the fingers.

As shown in FIGS. 11, 12 and 14, the head 133 of the plunger 30 is formed at its upper surface with an elongate vertical rod 134 in the shape of quadrangular prism. The elongate rod 134 extends upwardly from an eccentric position spaced apart from the central axis Y of the cylinder 110.

The adapter 121 is defined with a cavity 124 having a certain inner volumn extending upwardly from the lower end of the adapter 121. The adapter 121 has a conical inner wall 124a having a diameter decreasing gradually toward the upper side of the cavity 124. The cavity 124 has a central axis corresponding to the central axis Y of the cylinder 110.

According to its upward movement, the rod 134 of the plunger 130 comes into contact with the conical inner wall 124a of the adapter 121 at the upper end thereof. By virtue of the shape of the conical inner wall 124a, the upper end of the rod 134 is bent inwardly toward the central axis Y of the cylinder 110.

The cavity 124 of the adapter 121 is communicated with an elongate bore 121c positioned above the upper end of the cavity 124, thereby causing the injection liquid to flow from the cavity 124 to the elongate bore 121c.

The plunger 130 and adapter 121 are formed with coupling means, respectively. The coupling means allow the adapter 121 to be rotated and then moved downwardly in accordance with the successive rotating and downward movements of the plunger 130. The coupling means comprise grooves 126 defined on the inner surface of and adjacent to the lower end of the adapter 121 and a pair of protrusions 137 and 138 formed above the head 133 of the plunger 130 and adapted to be inserted into the grooves 126.

The cavity 124 is formed at its inner surface with four grooves 126 equally spaced from each other. A width t₁ of the respective grooves 126 has a value larger than a width t₂ of the respective protrusions 138 and 137 formed at the plunger 130, so the respective protrusions 138 and 137 are fitted into one of the grooves 126, respectively, in accordance with the upward movement of the plunger 130.

Each groove 126 is formed at its lower end with an annular step portion 121b′ extending inwardly from the flange 121b. The annular step portion 121b′ has a curved inner surface having a diameter increasing gradually toward its lower end. As the plunger 130 is pushed into the cylinder 110, although the protrusions 137 and 138 collide against the lower surfaces of the annular step portion 121b′, an extrusion 136 is bent slightly and then inserted smoothly into one of the grooves 126, respectively, while sliding along the curved inner surface of the annular step portion.

The head 133 of the plunger 130 is covered at its outer surface with an annular packing member 140.

Now, additional description related to the assembly process and operations of the safety syringe constructed according to the second embodiment of the present invention will be omitted because there is no difference between the first and second embodiments.

### INDUSTRIAL APPLICABILITY

As apparent from the above description, the present invention provides a safety syringe which is configured to effectively prevent an injection needle from moving unstably while in use. This is made possible by an adapter connected to the injection needle, which is threadedly coupled with the neck of a cylinder while interposing an O-ring therebetween. As a result, the injection needle can be pricked accurately at a desired position of the patient's body. In the safety syringe constructed according to the present invention, since the adapter is formed with grooves, whereas a plunger is formed with two protrusions diametrically opposite to each other so as to be inserted into the grooves, the adapter is rotated and then moved downwardly in accordance with the successive rotation and downward movements of the plunger after the coupling between the adapter and the neck of the cylinder is released. The plunger is formed at its upper surface with a vertical rod extending upwardly from an eccentric position, whereas the adapter is defined with a conical cavity. The upper end of the rod is inserted into the cavity while being bent inwardly. When the injection needle is withdrawn into the cylinder, the injection needle is firmly tilted in a lateral direction due to the strong restoring force of the rod so that it can be collapsed simply against the inner surface of the cylinder. In addition, a circular plate is formed at a certain lower position of the plunger. The circular plate enables the user's fingers gripping the safety syringe not to come into contact with the plunger to be pushed into the cylinder, thereby achieving the sanitary use of the safety syringe.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. a safety syringe comprising:
a cylinder defining a space for accommodating injection liquid and having a neck extending upwardly from an upper end thereof;
a plunger inserted inside the cylinder while being movable in the cylinder;
an adapter coupled to the neck of the cylinder; and
an injection needle connected to the adapter, wherein:
the plunger is provided at its upper surface with a vertical rod extending upwardly from an eccentric position with respect with a central axis of the cylinder;
the adapter is defined at its interior space with a cavity, into which the rod is inserted while being bent inwardly at its upper end in accordance with the upward movement of the plunger;
the adapter is threadedly coupled to the neck of the cylinder; and
coupling means are formed at the adapter and plunger, respectively, and adapted to allow the adapter to be rotated and then moved downwardly in accordance with the successive rotating and downward movements of the plunger, thereby causing the injection needle to be withdrawn into the cylinder.

2. The safety syringe as set forth in claim 1, wherein the adapter is coupled with a hub at its upper portion so that the hub is fitted in the adapter, and the injection needle is fixedly fitted in the hub.

3. The safety syringe as set forth in claim 1, wherein the injection needle is directly fixedly fitted in the adapter.

4. The safety syringe as set forth in claim 1, wherein the adapter is threadedly coupled with the neck of the cylinder at a certain portion spaced apart from a lower end of the adapter by a certain distance.

5. The safety syringe as set forth in claim 1, wherein the adapter and neck of the cylinder are formed, respectively, with helical threads to be threadedly coupled with each other.

6. The safety syringe as set forth in claim 5, wherein the helical threads are formed to have an inclination angle of 25° to 50° measured from a horizontal plane in a direction perpendicular to the central axis of the cylinder.

7. The safety syringe as set forth in claim 5, wherein the helical threads are formed to have an inclination angle of 30° measured from a horizontal plane in a direction perpendicular to the central axis of the cylinder.

8. The safety syringe as set forth in claim 5, wherein the helical threads are formed as two threads.

9. The safety syringe as set forth in claim 1, wherein the coupling means comprise a plurality of grooves defined at an inner surface of and adjacent to the lower end of the adapter, and protrusions extending upwardly and outwardly from an upper surface of the plunger and adapted to be inserted into the grooves of the adapter.

10. The safety syringe as set forth in claim 9, wherein each groove is formed at its lower surface with an inwardly-protruded annular step portion.

11. The safety syringe as set forth in claim 10, wherein the annular step portion has an inclined inner surface having a diameter increasing gradually toward its lower end.

12. The safety syringe as set forth in claim 10, wherein the annular step portion has a curved inner surface.

13. The safety syringe as set forth in claim 9, wherein the protrusions are formed at a lower end of the vertical rod extending upwardly from the eccentric position of the upper surface of the plunger.

14. The safety syringe as set forth in claim 9, wherein the protrusions are two protrusions diametrically opposite to each other.

15. The safety syringe as set forth in claim 14, wherein a first one of the protrusions is formed at the lower end of the vertical rod extending upwardly from the eccentric position of the upper surface of the plunger, and a second one of the protrusions is formed at a circular extrusion formed at the upper surface of the plunger diametrically opposite to the first one.

16. The safety syringe as set forth in claim 1, wherein the cavity defined in the adapter and adapted to allow the insertion of the rod has a central axis corresponding to the central axis of the cylinder, and is formed into a conical shape having a diameter decreasing gradually toward its upper side.

17. The safety syringe as set forth in claim 1, wherein the adapter is formed with a flange at its lower end to be coupled with the neck of the cylinder.

18. The safety syringe as set forth in claim 17, wherein an O-ring is inserted in the adapter so that it is located over an upper surface of the flange.

19. The safety syringe as set forth in claim 1, wherein the plunger is formed at its lower end with a knob, and the knob has a quadrangular contour with its respective side edges dented inwardly.

20. The safety syringe as set forth in claim 1, wherein the plunger is formed at its lower end with a knob, and also formed with a circular plate at a portion thereof spaced apart from the knob by a certain distance.
